(19) United States
(12) Patent Application Publication
(10) Pub. No.: US 2011/0195473 A1
(43) Pub. Date: Aug. 11, 2011

(54) METHOD AND DEVICE FOR PHOTOSYNTHESIS-SUPPORTED EXHAUST GAS DISPOSAL, PARTICULARLY CO2

(75) Inventor: Hermann-Josef Wilhelm, Kalkar (DE)

(73) Assignee: Maria Rogmans, Kalkar (DE)

(21) Appl. No.: 13/123,439

(22) PCT Filed: Oct. 7, 2009
(86) PCT No.: PCT/EP09/07179

§ 371 Date: Apr. 8, 2011

(30) Foreign Application Priority Data
Oct. 9, 2008 (DE) 10 2008 050 974.4
Oct. 24, 2008 (DE) 20 2008 014 199.0

(51) Int. Cl. C12P 3/00; C12M 1/04; C12M 1/00

(52) U.S. Cl. 435/168; 435/292.1; 435/289.1

(57) ABSTRACT

The invention relates to a method and to a device for photosynthesis-supported exhaust gas disposal, particularly CO₂ disposal. In order to compensate, even locally, for CO₂ produced by energy generation or by processing energy carriers, the invention proposes that exhaust gases from combustion processes or chemical processes act as a CO₂ source, wherein the exhaust gas is fed directly, or under pressure in water, forming carbon dioxide dissolved in water, into an at least partially closed system, in which rapidly growing photosynthetically active biomasses are cultivated, and that the biomass is harvested cyclically, and that further biomass reproduces automatically from the remaining biomass.

The invention relates to a method and a device for photosynthesis-supported exhaust gas disposal, particularly CO₂, according to the preamble of patent Claims 1 and 12.

In order to compensate for CO₂ contained in exhaust gases produced by the generation of energy, there is, or is to be, traded what is referred to as emission rights. In context with this consideration, an emission source that is being registered on a global scale, such as a coal-fired power station, is compensated by a source of energy generation pursuant to the Climate Convention such as a wind turbine or a biomass station, which generates carbon-neutral energy. In sum, it is intended to generate a portion of 25% of all generated electricity from regenerative energy by 2020. In other words, this means that CO₂ is produced somewhere in the world, and it needs to be transported by the atmosphere in order to be regenerated at another place, which means that it can be processed by biomass.

This is correct if viewed as a global sum. However, at a closer look it becomes obvious that the atmosphere needs to transport the emitted CO₂. Thus the atmosphere is also stressed in this compensated CO₂ consideration, at least during the transport. This already leads to negative climatic effects.

Methods and devices for introducing gas containing CO₂ as fertilizer into agricultural fields are known.

In this context it was shown that CO₂ has the effect of enormously increasing plant growth when introduced into the soil. In addition, aerial fertilization in greenhouses with CO₂, or gas containing CO₂, is known, which has also said growth-enhancing effect.

Likewise, it must be noted that large amounts of CO₂ are also generated in other processes, also in the preliminary stages during the generation of energy. This also applies to the field of renewable energies. When biogas is used, there is, on the one hand, the option to burn it at a relatively low methane content of only 50 to 55% and to generate energy. On the other hand, it becomes technologically more and more important to often post-treat biogas, which means to methanize it. This means that the original methane content is increased from 50% to up to 96%. Known methods are applied to achieve this aim.

As a considerable part of the residual gas in original biogas consists of CO₂, this naturally accumulates during methanization, i.e. during the post-treatment of biogas, in considerable amounts.

At the end, the biogas treated such possesses an achieved methane value of 96% and the same quality as natural gas, but, as mentioned before, CO₂ does accumulate in the meantime.

A further aspect is that the prices for fertilizers tend to show a clear increase. With respect to the cultivation of biomass for the generation of energy, the increasing prices for fertilizers lead to respective increases in cost, increasing the cost of what is referred to as the cultivation of energy crops.

Furthermore, WO/20071012313 discloses a greenhouse, in which several tiers are provided. It is proposed that in the lower tiers there is provided what is referred to as breeding areas, on which seedlings are bred. Later, the grown seedlings are rearranged to higher tiers. With respect to the breeding of new plants this may be advantageous, but for other applications this is unsuitable, because the rearrangement or transposition of the tiers requires a high functional, but also an energetic effort.

Such known concepts are applied at optimized growth conditions in a greenhouse. However, the individual approaches are useless, if the aim is to achieve the final effect of an optimized disposal of CO₂.

Therefore, the object of the invention was to further develop a process and a device such that amounts of CO₂ produced by the generation of energy or treatment of energy carriers at one location are also compensated near the same location.

The set objective is solved by a method of the generic kind according to the invention by the characterizing features of Claim 1.

Further advantageous embodiments of the method are indicated in the dependent Claims 2 to 11.

With respect to a device of the generic kind, the objective is solved according to the invention by the features of Claim 12.

Further advantageous embodiments of the device are indicated in the other dependent claims.

In the process part, the invention consists therein that exhaust gas from combustion processes or chemical processes act as a CO₂ source, wherein the exhaust gas is fed directly, or under pressure in water, forming carbon dioxide dissolved in water, into an at least partially closed system, in which rapidly growing photosynthetically active biomasses are cultivated, and that the biomass is harvested cyclically, and that further biomass reproduces automatically from the remaining biomass.

According to an advantageous embodiment, at least one part of the harvested biomass is recycled into said exhaust-producing energy generation process for the purpose of the generation of energy (biogas, dry fuel, bioethanol, biodiesel). In doing so, a closed substance cycle is formed, which even includes the CO₂ produced during the generation of energy.

According to a further advantageous embodiment, the carbonated water, which is aerated from exhaust gases, is fed in line with demand for the purpose of irrigating the biomass by monitoring a filling level at the feed-in point such that that as much water, which has been treated with carbonic acid and, if applicable, nutrient solutions, is added as can be absorbed and metabolized by the biomass. Thus both an optimized growth enhancement and a controlled harvestability of the biomass are provided.

According to a further advantageous embodiment, at least part of the biomass consists of duckweed, which floats in shallow tubs on top of said fed-in treated water. Duckweed is extremely strong and rapidly growing, and it produces large amounts of biomass in a short time. In doing so, it metabolizes CO₂ very well in this manner.

Alternatively, or additionally, the biomass may comprise wheat or similar germinating sees, which float in shallow tubs on top of said discharged treated water. Hereby special fleeces may be applied, to which the delicate roots can cling.

Duckweed and also germinating seeds are thus harvestable in very short periods.

These plants may also receive a post treatment in order to generate feedstuffs as an alternative to the energetic or chemical uses.

In total, plants are used, which do not only metabolize a significant amount of CO₂, but which also show a spectrum of ingredients, which allows to achieve optimum gas rates, for example, when dried or directly burned, or as a substrate for the generation of biogas.

The result of this is a functional synergy, in which, on the one hand, the biomass absorbs considerable amounts of CO₂, and, on the other hand, generates high-energy biomass.

Thus a considerable advantage results in the local arrangement of such a device according to the invention at the place of production of CO₂ exhaust gas, which stems from the utilization of exactly this same biomass. As a result, this is ultimately a closed-system energy generation process with an approximately permanent neutralization of CO₂.

According to a further advantageous embodiment, at least part of the biomass comprises cress (germinating seed), which floats in shallow tubs on top of said discharged treated water, or in said fleece material, respectively.

According to a further advantageous embodiment, harvesting of the biomass is carried out such that the increasing population of the biomass in the respective tubs, which are spatially limited, causes a lateral dropping out of excess biomass over a lowered rim of the tubs after a growth time, and that such biomass drops onto a conveyor system in a controlled manner and is transported to a location of processing. This simple principle, generally, exploits the “controlled proliferation” of the biomass. Automatic harvesting can also be carried out by simple tilting of the tubs, so that together with drained water also a respective amount of biomass, for example, duckweed, drops onto a conveyor belt and is transported out of the system.

According to a further advantageous embodiment, several tiers of tubs are stacked on top of another, provided that sufficient incidence of light is guaranteed for the purpose of photosynthesis. This results in a compact form of construction, wherein a maximum metabolic rate of CO₂ takes place in a given space, and a spatially optimized amount of usable biomass is created.

In doing so it was observed that duckweed reproduces already at 50 lux. Or in other words, already at a luminous/light intensity of 50 lux reproduction is not Zero any more. The upper limiting value is at approximately 3500 lux. Duckweed would burn at a higher luminous intensity.

According to a further advantageous embodiment, in order to artificially extend day light conditions, the plants are supplied with light, particularly UV light, in dark phases. A light spectrum with a wavelength from ca. 450 nanometres to 700 nanometres has proven to be optimal and contains all important wavelength portions for an optimum photosynthesis. In doing so, growth periods and thus also CO₂ active metabolism phases are extended also into the night time, whereby the efficiency of such a process and of such a device can be considerably increased.

According to a further advantageous embodiment, additionally or alternatively, for water supply with said treated water having high amounts of carbon dioxide dissolved in water, same is fed into said at least partially closed system by cold fogging. To this end, wetted water enriched with carbonic acid is introduced into the gas room, but not into the liquid of the partially closed system. During this process, the leaves of the biomass plants are also absorbing CO₂.

Partially closed system in this context means that the room around the plants is surrounded by walls, which are particularly permeable to light or light active. However, gases can be discharged in a controlled manner. This means that non-metabolized excess CO₂ on the one hand, but also oxygen generated by plants by photosynthesis on the other can escape in a controlled manner, for example, via adjustable valves or flaps.

According to a last embodiment according to the process, the process is applied underground in mines or underground caverns, wherein exhaust gas or CO₂ fed in or exhaust gas generated underground is collected and transported in the described manner into containers, which are filled with biomass and artificially illuminated, for a photosynthesis-supported metabolization of CO₂. This advantageous embodiment corresponds with the particular problem, which currently relates to the technological discharge of CO₂ by pumping it into the ground. The current experimental pumping of exhaust gas containing CO₂ or of CO₂ contained in exhaust gas into the ground carries the risk that the CO₂ enclosed there may be released by geothermal or seismic influences in phases, which may happen in large amounts. In this embodiment, in caverns located near such deposits that were pumped with CO₂ such as caves or mines, the constantly rising CO₂ is metabolized by the biomass in the described manner and thus permanently bound. The biomass obtained and fertilized with CO₂ is subsequently transported upward and processed there as source of protein, etc., or as a biogas substrate. By means of this generation of energy, the underground illumination for the photosynthesis can be operated. Thus also this energy input remains CO₂ neutral and the device is thus financing itself by means of the ingredients and/or excess energy.

With respect to a device of the generic kind, the core of the invention consists therein that a partially closed system is created in the form of a greenhouse comprising several tiers, in which rapidly growing photosynthesis-active biomass is cultivated in shallow containers and that, accumulating exhaust gases from a combustion or a chemical process act as a source of CO₂, which can be introduced by means of a pressure accumulator into water for the production of carbonated water, and such water can be fed into the shallow containers in the amount the water was absorbed by the biomass via a control device.

According to a further advantageous embodiment, the tubs in the tiers are arranged in an at least partially offset relationship for an improved constant supply of light. This ensures that a multitude of tiers can be placed on top of one another and still be supplied with an optimum amount of light, in the amount that, in the worst case, photosynthesis is barely maintained.

According to a further advantageous embodiment, the partially closed system comprises one or more valve or flap devices, via which excess gas—both oxygen and non-metabolized gas—can be discharged in a controlled manner. In doing so, the fact that oxygen produced by photosynthesis can also escape, is accounted for. This also applies to the possibly remaining small portion of non-metabolized CO₂. This, on the one hand, prevents an accumulation of pressure, which is harmful for the plants, and, except that, guarantees that there is always an optimally high portion of CO₂ for the growth and metabolism of the plants in what is formed and referred to as growth room.

According to a further advantageous embodiment, the roof and/or all lateral walls are embodied as pyramids or pyramidal body. Thus a simple form of construction is obtained, on the one hand, with a maximum light incident surface on the other. Thus the area can be used optimally by stacking the tubs in tiers while also an optimal light incident surface for daylight is created. In addition, the pyramids, which are flooded with light, are integrated into a landscape in a particularly ecologically responsible manner. Furthermore, wind hitting on the sides slides down optimally at all sides so that this benefits the statics in the construction of high pyramids of this kind.

Besides the pyramidal form it is, naturally, possible to also use other forms of construction allowing a high incidence of light, such as, for example, also a round area with a conic roof, or an elliptic area with a respective tapered roof.

According to a further advantageous embodiment, an additional illumination device is provided, by means of which light, particularly UV-rich light, can be supplied also at night time. In doing so, the growth cycle and thus both mass yields and cycle times are optimized during the automatic reproduction of the plants.

According to a further advantageous embodiment, the illumination device is supplied from an accumulator powered with electrical current generated from solar electricity or from the exploitation of residual heat. In this manner, also the supporting operation with artificial light remains CO₂ neutral.

According to a further advantageous embodiment, the partially closed system is embodied as a transportable container, which comprises a light-permeable material, particularly permeable to light or to UV light, at least at the roof side.

In this context it should be added that the definition of permeable to light or to UV light also comprises the spectrum of a wavelength from 450 to 700 nanometres.

According to a further advantageous embodiment, the container, or at least the wall and roof components permeable to light, are folding/collapsible in the form of a folding transport container for the purpose of transport of same and are unfolding on-site for their intended use.

According to a further advantageous embodiment, also the device for the generation of energy, or biogas, or the device for the generation of bioethanol, as well as one or more pressure accumulators are each stored in transportable containers.

According to a further advantageous embodiment, the partially closed system is arranged in a stationary room, which is permeable to light, particularly to UV light, of the type of a mobile or stationary greenhouse.

According to a further advantageous embodiment, the partially closed system, which means the device, is lowered into a hole dug into the soil of an agricultural field and covered from above with a roof permeable to light, particularly UV light, or a foil permeable to light, particularly UV light.

According to a further advantageous embodiment, the roof is embodied in a pyramidal form.

According to a last advantageous embodiment, the device for the disposal of CO₂ or exhaust gas containing CO₂ is located in an underground cavern or a mine. Thus the process according to Claim 11 is put into practice.

It is furthermore embodied that the tubs are embodied as bodies having a polygonal cross-section, are rotatable around an axis, and can be opened, and the biomass, for example, duckweed, can be taken out with a scraper. In addition, panel shaped hollow bodies can be formed, and rotation causes mature duckweed to adhere to the lateral walls after rotation, which can be scraped off with a scraper.

Furthermore it is advantageously embodied that the tubs are equipped with a light sensor on their inside such that by means thereof the obtainment of a surface fully covered by biomass or, respectively, duckweed, is recordable, and harvesting can be commenced. It has been observed that, if duckweed covers the whole surface, its further reproductive growth is declining. This is also referred to as growth depression.

This particular embodiment consists in the fact that the device comprises a greenhouse with planting tubs or planting containers, which are cultivated with aquatic plants or marsh plants that act as biomass, and that, for purposes of water supply, a supply of water from hot springs, and/or industrial waste waters, and/or sewage water, and/or mining water is provided. By feeding in such waters, thermal energy, on the one hand, and usable chemical ingredients are supplied on the other. The use of aquatic plants and the addition of said “waste waters” automatically cause a supply of fertilizers. In addition, the waters, which also carry thermal energy, cause that an all-year growth cycle is achieved, thus obtaining high yields of biomass from the aquatic plants year round.

In total, energetically and chemically valuable biomass is obtained from waste waters.

According to a further advantageous embodiment, the planting tubs or planting containers are arranged in a multitude of levels in a shelving or rack system. As a result, the effective cultivation area of the basic area of the greenhouse multiplies.

According to a further advantageous embodiment, the greenhouse comprises a cored factory building, or a cored skyscraper, or a cored cooling tower of a power station, or a cored water tower, which is equipped with glass or light-permeable foil. This way, existing industrial ruins can be cored and returned to a new valuable use.

According to a further advantageous embodiment, the greenhouse comprises a cylindrical building or a building of a polygonal cross-section, which is equipped with a light-permeable foil or glass, and which surrounds the tower of a wind turbine. In this example, the high towers of wind turbines are used in a highly efficient manner, wherein the greenhouses according to the invention quasi gain height and stand up by leaning on them.

This requires only a small area. What is decisive herein is the volume of the attainable height. This way, surrounding agricultural fields are not affected at all from the beginning. The renewable energy available by wind turbines may partially be used for automatic harvesting operations and treatment processes of the biomass in these greenhouse towers.

According to a further advantageous embodiment, the device is located in a direct proximity to a thermal spring, or an industrial plant, or a sewage treatment plant, or a mine. In these locations, said water is available at a short distance.

According to a further advantageous embodiment, the greenhouse is embodied as a pyramid, or a pyramidal body, or a cuboid.

According to a further advantageous embodiment, scraping elements, or an air-jet arrangement referred to as air broom is provided for the automatic harvesting of the biomass, which scrapes the biomass off the tubs or planting containers or expels it by specific application of compressed air in order to transport the biomass to a conveyor system.

According to a further advantageous embodiment, the device comprises a device for the production of biogas or bioethanol, or for the production of hydrogen, wherein energy carriers are producible from the harvested biomass, and the exhaust gases and/or the waste water and/or the waste heat can be fed back into the greenhouse.

According to a further advantageous embodiment, the device for the production of biogas and/or bioethanol is directly integrated, or implemented, into the device for the production of biomass.

According to a further advantageous embodiment, the exhaust gases of the device for the production of biogas and/or bioethanol can be fed into the greenhouse by means of exhaust gas recirculation in addition to the CO₂-rich aerial fertilization of the biomass.

According to a further advantageous embodiment, within the greenhouse there are arranged one or more tanks for the breeding of fish, into which the water/wastewater, which was initially fed through the planting tubs or planting containers can be fed, and vice versa.

With respect to a possible further use, the method or the device, respectively, is used for the operation of a clarifier of a sewage treatment plant. Thus the CO₂ obtained in clarifiers, stirring tanks and settling tanks is immediately biologically bound in the duckweed or the biomass, respectively.

A further use relates to mines or geological caverns, into which CO₂ is pressed, which means that the method and/or the device is applied for the CO₂ degasification of a mine, particularly a coal mine, wherein the accumulating CO₂-containing exhaust gas is collected and fed into water under pressure forming carbonic acid, and the carbonated water is used for fertilization purposes.

A last use relates to the disposal of CO₂ in residential buildings, wherein the method and/or the device is used such during the formation of exhaust containing CO₂ in the heating systems of residential buildings that the exhaust gas is fed into water under pressure forming carbonated water and transported away in pressure pipelines for further use.

Embodiments of the invention are shown in the Figures.

There are shown:

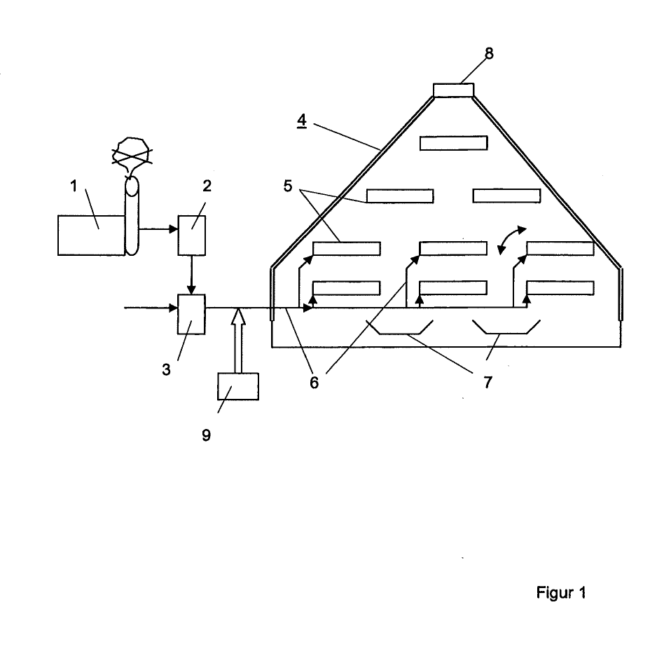

FIG. 1 General structure

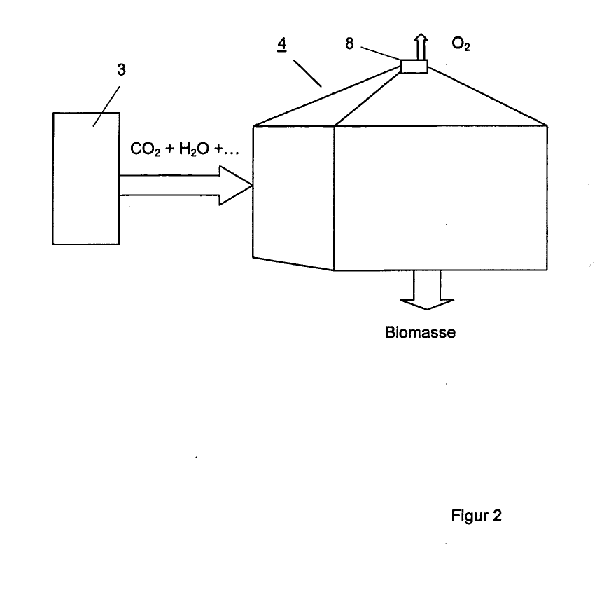

FIG. 2 Application as a transportable system

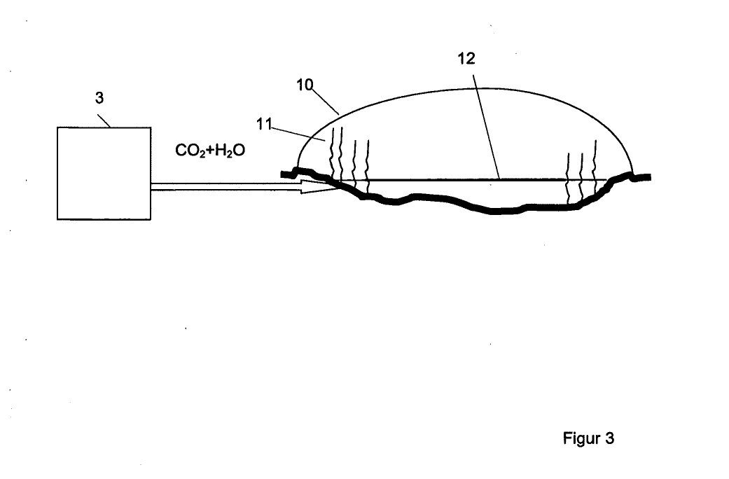

FIG. 3 Application in hollows

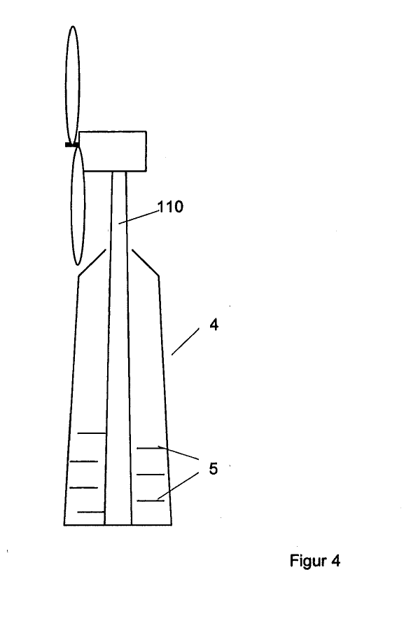

FIG. 4: Embodiment of a greenhouse tower around the tower of a wind turbine.

In principle it also applies to the above that in case of feeding in pressurized CO₂ into water, essentially carbonic acid dissolved in H₂O, i.e. H₂CO₃ in water is obtained. Thus the CO₂ becomes what is referred to as dissolved carbonic acid in case of said feeding into water under pressure.

According to the invention, CO₂ from EXHAUST GASES is used for the production of carbonic acid-rich water, which then acts as fertilizer for the biomass.

Regular harvesting takes place when duckweed or what is referred to as Wandering Sailor is used. In the case of duckweed this happens every 5 to 14 days.

FIG. 1 shows a first general form of embodiment. The exhaust gases of an exhaust plant or industrial plant 1 are not sent through the chimney, but firstly through a gas washer 2. Subsequently, the exhaust containing CO₂ is fed into a pressure accumulator 3, into which water is added under pressure from approx. 1 to 10 Bar forming carbon dioxide dissolved in water. During this process, the portion of CO₂ is adjusted from 0.05 to 0.5 grams per litre water, because this range of values is an optimal fertilizer and excludes an acidification of the biomass at the same time. Afterwards, the carbonated water obtained from exhaust gas is fed via a pipeline system 6 into said tubs 5. Thereby the filling level is controlled such that only that much water is supplied as is used, evaporates, or, if applicable, is metabolized by the plant. The tubs are hereby arranged in a partially closed system, which comprises a light-permeable wall 4. In this context, this system is, for example, embodied pyramidally as shown in this Figure, so that an optimal light-active surface for said photosynthesis is obtained. In addition, carbonated water is wetted within this system simultaneously, so that carbonic acid again degases as CO₂ (because this process is reversible), and CO₂ is offered in addition as an aerial fertilizer in this biomass-filled room. In this context, tubs 5 are, for example, tilting, so that, if this surface has formed a closed, for example, duckweed, mat, same may partially be poured off by tilting. To this end, on the ground of tubs 5 there is arranged a light sensor each, which is almost completely darkened the moment the surface is completely grown over and must be harvested.

Below there is a conveyor system, so that the poured off duckweed may be collected automatically and be transported away for further use.

On the top of the pyramidal body, which forms the partially closed system, a discharge flap 8 or a discharge valve is arranged in order to discharge excess gas, i.e. also oxygen produced by photosynthesis, at the top.

In addition, waste waters from an industrial plant 1 are, if applicable, pre-filtered in a filter 2, and, if applicable, but not necessarily, fresh water 3 is added and fed into the planting tubs 5 within the greenhouse. This is carried out by a pipeline system 6.

Likewise, thermal water or mining water from mines can be fed in. Besides the supply of these waters, obviously also heat is added, because these waters may obviously be tempered.

The biomass obtained after a particular growth period of several days can thus be harvested via scrapers or air brooms, during the process of which the biomass drops onto conveyor belts 7.

FIG. 2 shows a form of embodiment, wherein a system working as a biological CO₂—catalytic converter as container, particularly as transportable container, is used. This serves a mobile application.

Container 4 may in this context even consist of folding wall elements. Also here obtained rapidly growing biomass (duckweed) is removed. Herein, the exhaust gas may stem from stationary, but also mobile, producers of exhaust gas.

FIG. 3 shows an embodiment, wherein the process is applied in a hollow or a lake. Herein the biomass 12 is mainly produced from duckweed existing on top of the water surface and limited by reed-like plants 11 on the rim. The carbonated water formed according to the invention is hereby fed into the lake and degases there by releasing pressure in the same manner as in the partially closed systems referred to above, thus causing a considerable enhancement of growth. In this process, the lake or the hollow, respectively, is covered by a light-permeable (as described above) foil 10, in order to create a partially closed system also by this means. This embodiment is similar to a biotope and, on the one hand, binds CO₂ from exhaust gases in the same manner by extremely fast-growing biomass, and the produced biomass can, on the other hand, be harvested, i.e. collected, in said short time periods also here and returned to a further use accordingly.

Particularly this embodiment can also be used in clarifiers in sewage treatment plants, as already mentioned above.

FIG. 4 shows a form of embodiment, wherein the greenhouse according to the invention was constructed around the tower 110 of a wind turbine 100. Herein, the greenhouse 4 is very tall and erect, and the planting containers or planting tubs stacked in tiers are arranged inside. Thus only a comparably small basic area is required, but a large usable volume is created. Also the incident of light is optimal at this highly erect form of construction. By means of an optimal use of light, an optimal growth is achieved.

### REFERENCE SIGNS

1 Exhaust gas device
2 Gas washer
3 Pressure accumulator
4 Light permeable/UV light permeable wall
5 Planting tubs
6 Carbonated water
7 Conveyor system for biomass
8 Discharge flap/valve
9 Pipeline for feeding in mining or thermal water
10 Foil, light-/UV-light permeable
11 Plants growing on the sides
12 Duckweed
100 Windkraftanlage
110 Turm der Windkraftanlage

1. A method for the biological and ecologically compatible processing of CO₂ into carbon and oxygen, wherein exhaust gases from combustion processes or chemical processes act as a source of CO₂, wherein the exhaust gas is fed directly, or under pressure in water, forming carbon dioxide dissolved in water in an at least partially closed system where rapidly growing photosynthesis-active is cultivated, and the biomass is harvested cyclically, and further biomass reproduces either automatically from remaining biomass, or is cyclically refilled.

2. A method according to claim 1, wherein at least part of the harvested biomass for the generation of energy (biogas, dry fuel, bioethanol, biodiesel) is recycled into the said exhaust-producing energy generation process.

3. A method according to claim 1, wherein the carbonated water enriched from exhaust gases is fed in according to demand in order to supply water to the biomass, wherein a filling level is monitored at the feed-in point such that exactly the same quantity of treated water is supplied as is absorbed and metabolized by the biomass.

4. A method according to claim 1, wherein at least part of the biomass comprises duckweed, which floats in shallow tubs on said fed-in treated water.

5. A method according to claim 1, wherein at least part of the biomass comprises wheat or similar germinating seeds, which float in shallow tubs on said fed-in treated water.

6. A method according to claim 1, wherein at least part of the biomass comprises cress, which floats in shallow tubs on said fed-in treated water.

7. A method according to claim 4, wherein harvesting of the biomass is carried out such that the increasing population of the biomass in the respective tubs, which are spatially limited, causes a lateral dropping out of excess biomass over a lowered rim of the tubs, which in this process drops onto a conveying system in a controlled manner and is transported away for processing.

8. A method according to claim 1, wherein several tiers of tubs are stacked on top of one another, provided that sufficient incident of light allowing photosynthesis is guaranteed.

9. A method according to claim 1, wherein light, particularly UV-light, is supplied to the plants in order to artificially extend the supply of daylight in dark phases.

10. A method according to claim 1, wherein after additional or alternative water supply with said treated carbonated water, same is fed into said at least partially closed system by means of cold fogging.

11. A method according to claim 1, wherein the method is applied underground in mines or underground caverns by collecting exhaust gas or CO₂ there, which had been fed in or come into existence underground, and fed into the containers filled with biomass and artificially illuminated in the manner described for photosynthesis-supported CO₂ metabolization.

12. A device for the biological processing of CO₂ into carbon and oxygen for the carrying out of the method according to claim 1, wherein a partially closed system in form of a greenhouse comprising several tiers is formed, wherein rapidly growing photosynthesis-active biomass is cultivated in shallow containers, and that exhaust gases from combustion processes or chemical processes act as a CO₂ source, wherein the exhaust gas can be fed directly, or under pressure in water, forming carbon dioxide dissolved in water, and that said water can be fed into said shallow containers via a control device in the amount of the water absorbed by the biomass.

13. A device according to claim 12, wherein the tubs in the tiers are arranged in an at least partially offset relationship for an improved constant supply of light.

14. A device according to claim 12, wherein the partially closed system comprises one or more valve or flap devices, by means of which excess gas—oxygen and non-metabolized gas—can be discharged in a controlled manner.

15. A device according to claim 12, wherein the roof and/or all lateral walls are embodied as pyramid or pyramidal body.

16. A device according to claim 12, wherein an additional illumination device is provided, by means of which light, particularly UV-rich light, can be supplied also at night time.

17. A device according to claim 12, wherein the illumination device is fed from an accumulator with electricity obtained from solar power or from the exploitation of residual heat.

18. A device according to claim 1, wherein the partially closed system is embodied as a transportable container, comprising light-permeable, particularly UV-light permeable, material at least on the roof side.

19. A device according to claim 18, wherein the container or at least the light-permeable wall and roof components are folding/collapsible in the form of a folding transport container for the purpose of transport of same and are unfolding on-site for their intended use.

20. A device according to claim 18, wherein also the device for the production of energy, or the device for the production of bioethanol, and one or more pressure accumulators are each housed in transportable containers.

21. A device according to claim 12, wherein the partially closed system is arranged in a stationary, light-permeable, particularly, UV-light permeable, room of the type of a mobile or stationary greenhouse.

22. A device according to claim 12, wherein the partially closed system, i.e. the device, is lowered into a dug hollow in an agricultural field and covered from above with a light-permeable, particularly, UV-light permeable, roof, or a light-permeable, particularly UV-light permeable, foil.

23. A device according to claim 12, wherein the roof is embodied pyramidally.

24. A device according to claim 12, wherein the device for the disposal of CO₂ or of exhaust gases containing CO₂ is located in an underground cavern or in a mine.

25. A device according to claim 12, wherein the tubs are embodied as bodies with a polygonal cross-section, which are rotatable around an axis and which can be opened, and that the biomass, for example, duckweed, can be collected with a scraper.

26. A device according to claim 12, wherein the tubs are equipped with a light sensor on the inside such that it is possible to record the achievement of a surface closed by biomass or duckweed, respectively, and harvesting can be commenced.

27. A device according to claim 12, wherein the device comprises the greenhouse (4) with planting tubs or planting containers, which are planted with aquatic or marsh plants as biomass, and that for purposes of water supply, a supply of water from thermal springs and/or industrial waste water and/or sewage water and/or mining water is provided.

28. A device according to claim 27, wherein the planting tubs or planting containers are arranged in a multitude of tiers in a shelving or rack system.

29. A device according to claim 27, wherein the greenhouse comprises a cored factory building, or a cored skyscraper, or a cored cooling tower or a power station, or a cored water tower, which are equipped with glass or light-permeable foil.

30. A device according to claim 27, wherein the greenhouse comprises a cylindrical construction, or a construction having a polygonal cross-section, which is equipped with light-permeable foil or glass, and which surrounds the tower of a wind turbine.

31. A device according to claim 27, wherein the device is located in direct proximity of a thermal spring, or an industrial plant, or a sewage treatment plant or a mine.

32. A device according to claim 27, wherein the greenhouse is embodied as a pyramid or a pyramidal body or a cuboid.

33. A device according to any of the claim 1, wherein scraping elements, or an air-jet arrangement referred to as “air broom” is provided for the automatic harvesting of the biomass, which scrapes the biomass off the tubs or planting containers, or expels it by specific application of compressed air in order to transport the biomass to a conveyor system.

34. A device according to claim 1, wherein the device comprises a device for the production of biogas, or a device for the production of bioethanol, or a device for the production of hydrogen, wherein energy carriers are obtainable from the harvested biomass, and the exhaust gases and/or the waste waters and/or the waste heat can be fed back into the greenhouse.

35. A device according to claim 1, wherein the device for the production of biogas and/or bioethanol is directly integrated or implemented in the device for the production of biomass.

36. A device according to claim 34, wherein the exhaust gases of the device for the production of biogas and/or bioethanol can be fed into the greenhouse in addition to the CO₂-rich aerial fertilization of the biomass by means of a device for feeding back the exhaust gas.

37. A device according to claim 1, wherein one or more fish breeding tanks are arranged within the greenhouse, into which the water/waste water can be fed, which was firstly transported through the planting tubs or planting containers, and vice versa.

38. A device for carrying out a method according to claim 1, for the biological processing of CO₂ into carbon and oxygen for the carrying out of the method wherein a partially closed system in form of a greenhouse comprising several tiers is formed, wherein rapidly growing photosynthesis-active biomass is cultivated in shallow containers, and exhaust gases from combustion processes or chemical processes act as a CO₂ source, wherein the exhaust gas can be fed directly, or under pressure in water, forming carbon dioxide dissolved in water, and that said water can be fed into said shallow containers via a control device in the amount of the water absorbed by the biomass are applied for the operation of a clarifier of a sewage treatment plant.

39. A device for carrying out a method according to claim 1 wherein the device is applied for the discharge of CO₂ from a mine, particularly a coal mine, wherein the obtained exhaust gas containing CO₂ is collected and fed into water forming carbonic acid under pressure, and the carbonated water is used as a fertilizer.

40. A device for carrying out a method according to claim 1, wherein the method and/or the device is used when exhaust gas containing CO₂ is obtained in the heating systems of residential buildings such that the exhaust gas is fed into water forming carbonic acid under pressure, and transported away for further use by means of pressure pipelines.
